(19) United States
(12) Patent Application Publication
(10) Pub. No.: US 2005/0010261 A1
(43) Pub. Date: Jan. 13, 2005

(54) Application of stimulus to white matter to induce a desired physiological response

(75) Inventors: Jurgen Luders, Cleveland Heights, OH (US); Imad Najm, Broadview Heights, OH (US); Hans O. Luders, Cleveland Heights, OH (US); Nicholas M. Boulis, Moreland Hills, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/819,749

(22) Filed: Apr. 7, 2004

Related U.S. Application Data
(63) Continuation-in-part of application No. 10/689,455, filed on Oct. 20, 2003.
(60) Provisional application No. 60/420,079, filed on Oct. 21, 2002.
(60) Provisional application No. 60/460,993, filed on Apr. 7, 2003.

(51) Int. Cl. A61N 1/18

(52) U.S. Cl. 607/45

(57) ABSTRACT

The present invention relates to providing a stimulus to brain structures having high fiber density, such as white matter tracts. The stimulus, which can be electrical, pharmacological and/or genetic, is operative to employ the white matter to affect associated brain structures associated with the white matter to help induce a desired physiological response, such as helping to reduce or control seizures.

### CROSS-REFERENCE TO RELATED APPLICATIONS

This is a continuation-in-part application of U.S. patent application Ser. No. 10/689,455, which was filed on Oct. 20, 2003, and entitled “ELECTRICAL STIMULATION OF THE BRAIN,” and claims the benefit of U.S. Provisional Application No. 60/460,993, which was filed on Apr. 7, 2003 and entitled “APPLICATION OF STIMULUS TO WHITE MATTER TO INDUCE A DESIRED PHYSIOLOGICAL RESPONSE,” each of which is incorporated herein by reference.

### TECHNICAL FIELD

The present invention relates to treatment for the nervous system, and more particularly to systems and methods for applying a stimulus to white matter of the brain to induce a desired physiological response.

### BACKGROUND

Presently, various different approaches exist to stimulate the brain to help alleviate degenerative diseases and nervous system disorders, such as Parkinson's disease and epilepsy. For example, electrical stimulation can provide an effective treatment for patients when surgical lesioning of brain tissue is not a suitable option as well as when patients are not sufficiently responsive to other treatment modalities, such as direct administration of pharmacological agents or drug therapy.

Some different types of electrical stimulation treatments include vagal nerve stimulation, cerebellar stimulation, and deep brain stimulation. One major advantage of electrical stimulation over lesioning procedures (e.g., pallidotomy and thalamotomy) is that the electrical stimulation can be reversible and adjustable. For example, brain stimulation can be implemented with no destruction of brain tissue and the stimulator can be removed, if needed. Additionally, the stimulation can be adjusted (e.g., increased, minimized or turned off or otherwise modified) to achieve better clinical effects for each patient.

Vagal nerve stimulation is one accepted type of treatment for epilepsy and Parkinson's disease. Vagal nerve stimulation is typically performed via a stimulator device, which includes a generator that electrically stimulates the brain through the vagus nerve to prevent seizures. The generator is surgically implanted into the chest, such as under the collarbone, and can be activated automatically or manually, such as by passing a magnet over the device.

In general, deep brain nuclei stimulation involves the precise electrical stimulation of specific deep brain structures using implanted electrodes. More recently, there has been significant work in the area of electrical stimulation of the subthalamic nucleus (STN) in which miniature electrodes are placed into the STN on one or both sides of the brain. STN is a structure located deep within the brain that has been found to control many aspects of normal motor function. Electrical stimulation of the STN effectively jams or blocks the abnormal circuitry of the brain, such as in the case of Parkinson's disease or epilepsy. While such direct electrical stimulation can be effective in many cases, it generally requires monitoring and control of the electrical stimulus being applied.

Another type of treatment for brain disorders and diseases includes gene therapy. In gene therapy, genes are delivered directly to selected target cells in the nervous system to modify phenotypic characteristics of the target cells, such as in a cytotoxic or restorative manner. Various delivery mechanisms have been developed to facilitate introduction of the DNA sequence of interest to the target cells. These include the use of both viral and non-viral vectors. Viral vectors including retroviruses, adenoviruses, adeno-associated viruses, and herpes viruses tend to have increased transfection rates when compared to non-viral techniques.

These and other gene therapy approaches operate by inducing expression of therapeutic proteins that provide a desired physiological response at the target cells. Because the proteins can be expressed intracellularly, cellular pathways can be manipulated in a manner not achievable via traditional oral or intravenous administration. A particular viral or non-viral approach can be selected for a given patient according to its unique properties and the patient's condition.

### SUMMARY

The present invention relates to delivery of a stimulus to white matter tracts in the brain to mitigate or help control seizures or provide other desired physiological results. The stimulus can be implemented as one or more of a genetic stimulus, a pharmacological stimulus or an electrical stimulus. The stimulus is delivered to appropriate cells of white matter and, in turn, is operative to induce a desired physiological response, such as in target brain cells that project into the white matter where the stimulus is delivered. For example, the physiological response can be characterized by altering phenotypes of the target cells to inhibitory or less excitatory. Alternatively, the phenotype of the target cells can be altered to more excitatory.

According to one aspect of the present invention, the stimulus can include delivering a vector to a white matter delivery site to induce desired gene-based neuromodulation. The vector, which can be a viral or non-viral vector, contains genetic materials coded to express a desired protein in target brain cells that project into the white matter delivery site. The vector can be selected to facilitate retrograde uptake from the white matter through connecting neural pathways into the target brain cells. Additionally or alternatively, suitable external means can be employed to facilitate transport of the vector (or at least the DNA contained therein) to the target cells.

In accordance with an aspect of the present invention, the white matter delivery site can include one or more of the fornix, corpus callosum and perforant pathways. The particular white matter structure and region to which the stimulation is applied can vary based on the location of the epileptogenic zone. According to one aspect of the present invention, an appropriate vector can be delivered to cells in the fornix, such as where the epileptic zone has been determined to include the hippocampus. According to another aspect of the present invention, a vector can be delivered to the corpus callosum, such as where the epileptic zone has been determined to be cortical. Similarly, the perforant path can be employed as the delivery site for affecting synaptic activity in the hippocampus in accordance with an aspect of the present invention.

To facilitate implantation of a suitable stimulus delivery device, endoscopic or stereotactic means can be utilized to efficiently place the delivery device in communication with the white matter delivery site in accordance with an aspect of the present invention. Because of the potentially chronic nature of such gene-based therapies, in some circumstances, it may be appropriate to employ electrical or another type of test stimulus to a selected white matter delivery site prior to the delivery of the vector at such location to help ensure the site is appropriate. Once the efficacy of the site has been adequately confirmed, the vector can be delivered accordingly.

In accordance with an aspect of the present invention, a stimulator that provides electrical stimulation can also be configured to deliver the vector to the delivery site. This may afford greater accuracy in locating an appropriate delivery site and facilitate delivery of the vector to appropriate white matter. For example, the stimulator location can be adjusted, if needed, to locate white matter that provides desired pathways with the target brain cells to further facilitate retrograde transport of the vector to such target cells.

### BRIEF DESCRIPTION OF THE DRAWINGS

The foregoing and other aspects of the present invention will become apparent to those skilled in the art to which the present invention relates upon reading the following description with reference to the accompanying drawings.

FIG. 1 is a block diagram illustrating system for delivering a stimulus to the brain in accordance with an aspect of the present invention.

FIG. 2 is an example of one type of system for delivering a stimulus to the corpus callosum in accordance with an aspect of the present invention.

FIG. 3 is another example of a system for delivering a stimulus to the fornix associated with an epileptogenic structure in accordance with an aspect of the present invention.

FIG. 4 is a schematic example depicting some neurological pathways of the brain associated with the hippocampus and fornix, which can be employed in stimulation in accordance with an aspect of the present invention.

FIG. 5 is an example of another type of system for delivering a stimulus to the corpus callosum in accordance with an aspect of the present invention.

FIG. 6 is an example of a system for supplying stimuli to plural white matter regions in accordance with an aspect of the present invention.

FIG. 7 is a coronal section of the brain illustrating corpus callosum stimulation in accordance with an aspect of the present invention.

FIG. 8 is a flow diagram illustrating a methodology for supplying a stimulus to the brain in accordance with an aspect of the present invention.

### DETAILED DESCRIPTION

The present invention relates to application of a stimulus to neurological structures having high fiber density, such as white matter, to induce a desired physiological response in associated target cells that project into the white matter where the stimulus is applied. The application of a stimulus to such white matter or like brain structures can help reduce seizures or otherwise help control neurological disorders or diseases by affecting the associated target cells in a desired manner.

FIG. 1 depicts a schematic example of a system 10 for delivering a stimulus in accordance with an aspect of the present invention. The system 10 includes a delivery device 12, at least a portion of which is operative to deliver a stimulus to selected white matter 14 of a patient's brain 16. White matter is generally formed of nerve fibers, called axons, which are insulated by a fatty substance known as myelin. White matter carries information between nerve cells of associated non-white matter brain structures, including gray matter, by conducting electrical impulses through pathways formed of nerve fibers.

The stimulus can include a genetic materials operative to induce expression of a protein that modifies the phenotypic characteristics of target cells that project into the white matter 14 where the stimulus is applied.

For example, the stimulus can include a viral or non-viral delivery vehicle, namely, a vector that carries genes or transgenes engineered to express a desired therapeutic protein or precursors to such protein. Such a genetic vector can be engineered induce expression of protein, such as an enzyme, capable of altering synaptic activity in a desired manner. Alternatively, the stimulus can include therapeutic doses of one or more pharmacological agents or a combination of pharmacological agents and genetic vector capable of inducing expression of one or more therapeutic proteins. Additionally, or alternatively, the stimulus may include an electrical stimulus individually or in combination with genetic and/or pharmacological stimuli.

In accordance with an aspect of the present invention, the stimulus can include vector that is applied to the white matter (e.g., by microinjection or other delivery mechanism) to induce a desired physiological response in target cells that project into the white matter. In order to facilitate retrograde transport of the vector from the white matter into the target cells, a vector (e.g., rabies virus or a pseudotype) designed for transport through intervening nerve fibers can be used.

As mentioned above, the vector can be viral or non-viral. The vector can include, for example, genes for precursors to neuropeptide transmitters or for enzymes that produce neurotransmitters. For instance, an appropriate viral (or non-viral) vector can be utilized for glutamate decarboxylase (GAD) gene transfer as a means of inducing or increasing the production of the inhibitory amino acid neurotransmitter GABA at desired target cells. The vector can be engineered to transfer the genes for green fluorescence protein (GFP) and GAD65 or GAD67, for instance. Those skilled in the art will appreciated that such an approach can provide an effective chronic treatment modality to enable neuroprotection, seizure prevention, neuromodulation or a combination thereof. It is believed that GAD vectors act therapeutically in a variety of disorders believed to result from excess synaptic excitation, including neurodegenerative disorders and epilepsy. GAD gene transfer can also be applied to focused neuromodulation in patients with Parkinson's disease.

Suitable vectors also can be employed to transfer genes for enzymes that produce dopamine according to an aspect of the present invention. This approach thus can be utilized as a means of replacing dopamine production for patients with Parkinson's disease. Additionally, it will be appreciated that the vectors can be designed by using tissue specific promoters so that the desired proteins are expressed in specific cells (e.g., brain cells).

Various techniques using viral vectors for the introduction of a desired gene (e.g., for GAD) into a target cell can be utilized in accordance with an aspect of the present invention. It is desirable to employ viral vectors that exhibit low toxicity to a host cell and induce production of therapeutically useful quantities of GAD protein in a tissue-specific manner. Viral vector methods and protocols that can be used in accordance with an aspect of the present invention are disclosed in Kay et al. Nature Medicine 7:3340, 2001. The use of specific vectors, including those based on adenoviruses, adeno-associated viruses, herpes viruses, and retroviruses are described below.

By way of example, the use of recombinant adenoviruses as gene therapy vectors is described in W. C. Russell, Journal of General Virology 81:2573-2604, 2000; and Bramson et al., Curr. Opin. Biotechnol. 6:590-595, 1995. Adenovirus vectors have desirable properties including: (1) they are capable of highly efficient gene expression in target cells and (2) they can accommodate a relatively large amount of heterologous (non-viral) DNA. One exemplary form of recombinant adenovirus is a “gutless, “high-capacity”, or “helper-dependent” adenovirus vector. Such a vector features, for example, the following characteristics: (1) the deletion of all or most viral-coding sequences (those sequences encoding viral proteins), (2) the viral inverted terminal repeats (ITRs), which are sequences required for viral DNA replication, (3) up to 28-32 kb of “exogenous” or “heterologous” sequences (e.g., sequences encoding GAD), and (4) the viral DNA packaging sequence which is required for packaging of the viral genomes into infectious capsids. For specifically targeting non-white matter brain structures, preferred variants of such recombinant adenoviral vectors contain tissue-specific (e.g., neuron) enhancers and promoters operably linked to the gene.

Other viral vectors that can be used in accordance with an aspect of the present invention are adeno-associated virus (AAV)-based vectors. MV-based vectors are advantageous because they exhibit high transduction efficiency of target cells and can integrate into the host genome in a site-specific manner. Use of recombinant MV vectors is discussed in detail in Sacchettoni S, Benchaibi M., Sindou M., Belin M., Jacquemont B., Glutamate-modulated production of GABA in immortalized astrocytes transduced by a glutamic acid decarboxylase-expressing retrovirus, GLIA 1998;22(1):86-93; Robert J., Bouilleret V., Ridoux V., Valin A., Geoffroy M., Mallet J., et al., Adenovirus-mediated transfer of a functional GAD gene into nerve cells: Potential for the Treatment of Neurological Disease, Gene Therapy 1997; 4(11):1237-1245; and in Mi J., Chatterjee S., Wong K., A J C., Lawless G., Tobin, Recombinant adeno-associated virus (MV) drives constitutive production of glutamate decarboxylase in neural cell lines. J. Neurosci. Res. 1999; 57:137-148. A suitable MV vector comprises a pair of MV inverted terminal repeats which flank at least one cassette containing a tissue (e.g., brain) or cell (e.g., neuron) specific promoter. The DNA sequence of the MV vector, including the ITRs, the promoter and GAD gene may be integrated into the host genome.

The use of herpes simplex virus (HSV)-based vectors is discussed in detail in Cotter and Robertson, Curr. Opin. Mol. Ther. 1:633-644, 1999. HSV vectors deleted of one or more immediate early genes (IE) are advantageous because they are generally non-cytotoxic, persist in a state similar to latency in the host cell, and afford efficient host cell transduction. Recombinant HSV vectors can incorporate approximately 30 kb of heterologous nucleic acid. Desired characteristics of a HSV vector include a vector that: (1) is engineered from HSV type 1, (2) has its IE genes deleted, and (3) contains a tissue-specific (e.g., brain) promoter operably linked to a GAD gene (e.g., GAD65 or GAD67). HSV amplicon vectors may also be useful in various methods of the invention. Typically, HSV amplicon vectors are approximately 15 kb in length, and possess a viral origin of replication and packaging sequences. For example, Wilson S., Yeomans S., Bender M., Lu Y., Goins W., Glorioso J., in Antihyperalgesic effects of infection with a preproenkephalin-encoding herpes virus. Proc. Natl. Acad. Sci. USA 1999; 96(6):3211-3216, have demonstrated that herpes simplex (HSV) vectors may be used to transfer the gene for preproenkephalin to spinal sensory neurons.

Retroviruses such as C-type retroviruses and lentiviruses might also be utilized in accordance with an aspect of the present invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a GAD65 gene, for example. In methods of delivery to white matter, in accordance with an aspect of the present invention, it may also encode an enzyme to produce or control the production of a neuron-specific receptor protein.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells. Lentiviral vectors for use in the invention may be derived from human and non-human (including SIV) lentiviruses. Examples of suitable lentiviral vectors include nucleic acid sequences required for vector propagation as well as a tissue-specific promoter, such as may be operably linked to a GAD65 of GAD67 gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping.” The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be utilized in accordance with an aspect of the present invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain alphavirus genetic elements required for RNA replication, and a heterologous nucleic acid such as one encoding a GAD protein. Within an alphivirus replicon, the heterologous nucleic acid may be operably linked to a tissue-specific (e.g., brain) promoter or enhancer.

Recombinant, replication-defective alphavirus vectors are advantageous because they are capable of high-level heterologous (therapeutic) gene expression, and can infect a wide host cell range. Alphavirus replicons may be targeted to specific cell types (e.g., neurons) by displaying on their virion surface a functional heterologous ligand or binding domain that would allow selective binding to target cells expressing a cognate binding partner. Alphavirus replicons may establish latency, and therefore long-term heterologous nucleic acid expression in a host cell. The replicons may also exhibit transient heterologous nucleic acid expression in the host cell. A preferred alphavirus vector or replicon is non-cytopathic.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the secretion of a GAD gene product from the host cell.

To combine advantageous properties of two viral vector systems, hybrid viral vectors can be used to GAD gene transfer to target tissue (e.g., brain). Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded MV genomes in adenoviral capsids containing a combination of MV and adenoviral ITRs may be used to transduce cells. In another variation, an MV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186,2000. Retroviral genomes contained within an adenovirus may integrate within the host cell genome and effect stable GAD gene expression.

In addition to viral vector-based methods, non-viral methods can also be used to introduce a predetermined gene into a target cell in accordance with an aspect of the present invention. A disclosure of non-viral methods of gene delivery is provided in Nishikawa and Huang, Human Gene Ther. 12:861-870, 2001. One example of a non-viral gene delivery method, according to the invention, employs plasmid DNA for GAD gene transfer into a target cell. Plasmid-based gene delivery methods are generally known in the art and are described in references, such as Ilan, Y., Curr. Opin. Mol. Ther. 1:116-120,1999, Wolff, J. A., Neuromuscular Disord. 7:314-318,1997 and Arztl, Z., Fortbild Qualitatssich 92:681-683, 1998. Alternatively, other non-viral vector, such as including fibroblasts, stem cells, astrocytes and immature neurons also could be utilized in accordance with an aspect of the present invention.

Synthetic gene transfer molecules can be designed to form multimolecular aggregates with plasmid DNA (e.g., harboring a GAD coding sequence operably linked to a brain-specific promoter). These aggregates can be designed to bind to a target cell (e.g., neuron) surface to affect synaptic activity in a desired manner. Cationic amphiphiles, including lipopolyamines and cationic lipids, may be used to provide receptor-independent GAD gene transfer into target cells (e.g., neurons). In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139,1995 and Lasic and Templeton, Adv. Drug Delivery Rev. 20:221-266,1996. For gene delivery, DNA may also be coupled to an amphipathic cationic peptide (See, e.g., Fominaya et al., J. Gene Med. 2:455464, 2000).

Methods that involve both viral and non-viral based components can also be used according to an aspect of the present invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

DNA microencapsulation can also be used to facilitate GAD gene transfer in accordance with an aspect of the present invention. Microencapsulated gene delivery vehicles may be constructed from low viscosity polymer solutions that are forced to phase invert into fragmented spherical polymer particles when added to appropriate nonsolvents. Methods involving microparticles are discussed in Hsu et al., J. Drug Target 7:313-323,1999 and Capan et al., Pharm. Res. 16:509-513, 1999.

Methods involving microencapsulated recombinant cells may be used in the invention. Such an approach may be used in either in vivo or ex vivo techniques. Cells that contain an expression vector coding for GAD or that have been engineered to stably express GAD may be encapsulated in microcapsules that provide protection from immune mediators and allow appropriate release of the GAD protein. Preferred microencapsulation particles, also referred to as encapsulation devices, consist of biocompatible and biodegradable components. Techniques involving microencapsulated cells are discussed in Ross et a!. Hum. Gen. Ther. 11:2117-2127, 2000 and in Fong et al., Crit. Rev. Ther. Drug Carrier Syst. 17:1-60, 2000.

Those skilled in the art will understand and appreciate that protein transduction offers an alternative to gene therapy for the delivery of a stimulus, such as a protein, into target cells, in accordance with an aspect of the present invention. Protein transduction is the internalization of proteins into a host cell from the external environment. The internalization process relies on a protein or peptide which is able to penetrate the cell membrane. To confer this ability on a normally nontransducing protein, the non-transducing protein can be fused to a transduction-mediating protein such as the antennapedia peptide, the HIV TAT protein transduction domain, or the herpes simplex virus VP22 protein. See Ford et al., Gene Ther. 8:1-4, 2001.

Those skilled in the art will appreciated that these and other vectors are generally capable of transduction of pre-synaptic neurons, such that a substantial portion of the vector can be transported to the target cell bodies. Those skilled in the art will understand and appreciate mechanisms, which can be pharmacological, electrical or gene-based, that can be utilized to facilitate transport of the injected vector (or portions thereof) from the white matter to the target brain cells.

As mentioned above, the vectors include genetic material such as an expressible DNA sequence that encodes a native or non-native polypeptide, such as a therapeutic protein. The DNA sequence can be of any length and can include genomic DNA fragments, engineered DNA produced in a microbial host, or synthetic DNA. While much of the foregoing has related to GAD gene transfer, those skilled in the art will appreciate other suitable proteins that can be expressed. That is, because a given vector is engineered to include specific genes or transgenes, a vector can be utilized to express virtually any desired therapeutic protein in the target cells of the brain 16. Those skilled in the art will further understand and appreciated various elements (e.g., promoters, terminators, transcription- and translation-regulating sequences, and the like) that can be employed to provide an expressible genetic construct for a given vector.

There exist an assortment of delivery devices 12 that can be employed to deliver the vector at a desired white matter delivery site. For example, the delivery device 12 can include a hollow microneedle having an opening at its distal end through which the vector solution can be injected. A delivery system 18 is coupled to control delivery of the vector via the device. 12. The delivery system also may include a source 20 of vector-containing solution that is in fluid communication with the needle, such as through an interconnecting conduit.

By way of example, the source 20 can be a receptacle, such as a syringe, which holds the solution comprising the genetic material. Upon activation, the solution flows from the source 20 through the hollow center of the microneedle where it is jetted from the distal tip of the microneedle.

Alternatively, a solid microneedle can be mounted for longitudinal movement within a tube. One or more channels operatively associated with the tube can be employed as a pathway for delivering the vector solution that contains the genetic material to the designated delivery site. The channels can be separate from and attached to the needle tube, or can be formed directly in the needle.

As described above, the delivery site in the white matter 14 provides a pathway for transporting the vector from the delivery device 12 to the target cells, such as the epileptogenic focus. The delivery device 12 can be positioned adjacent to, in contact with or within a selected white matter structure 12, such that the vector can be injected into white matter cells. Where more than one epileptogenic focus exists, multiple delivery devices (e.g., microneedles systems) can be utilized to inject appropriate vectors, which can be the same or different vectors, for example, into white matter structures associated with each respective focus in accordance with an aspect of the present invention. Alternatively, the same delivery device can be used to supply vectors at multiple white matter delivery sites. For example, delivery devices can be used unilaterally, such as where a focus exists only in a single hemisphere of the brain 16, or bilaterally, such as where foci exist in both hemispheres.

The delivery device 12 is positioned (e.g., by stereotaxis or endoscopy) relative to the white matter 14 so as to enable microinjection. A micromanipulator (e.g., mechanical or computer controlled) further can be employed to place the delivery device at the desired delivery site or plural devices at determined sites in the white matter. The micromanipulator or micropositioner is a highly precise instrument that facilitates positioning a microneedle, micropipette, or other microtool in the field of a microscope within the area to be worked upon. Because the delivery device, such as including a microneedle, is being inserted into white matter, a micromanipulator in conjunction with appropriate endoscopic means can enable microinjection in a precise location for delivering a defined distribution of the vector at predetermined anatomic coordinates of selected white matter.

The delivery device is utilized to supply a therapeutically effective amount of the vector. It will be appreciated that a therapeutically effective amount is an amount which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. By way of general example, the microinjection may include approximately a 10 to 5000 nl injection of 3×10¹⁰ pt/ml of the vector operative to induce expression of the desired therapeutic protein or a precursor thereof, such as mentioned above.

Various diagnostic techniques can be utilized, individually or in combination, to determine the location of one or more epileptogenic foci (or zones) for a patient. Some examples include electroencephalography (EEG), magnetic resonance imaging (MRI), positron emission tomography (PET), single photon emission computed tomography (SPECT), magnetoencephalography (MEG), magnetic resonance spectroscopy (MRS), depth electrode and subdural grid implantation, video monitoring, neuropsychological testing and so forth. Those skilled in the art will understand and appreciate other types of diagnostic techniques that can be utilized to help ascertain epileptogenic zones in a patient. Such an approach, for example, including subdural electrode grid visualization, can be integrated to provide a detailed three-dimensional reconstruction of the patient's brain identifying the epileptogenic focus (or foci). Such techniques can also be employed further to correlate data and approximate with a high degree of precision a delivery site in the white matter into which the ascertained epileptogenic focus projects.

By way of example, some epileptogenic structures include the hippocampus, STN and neocortical structures. According to an aspect of the present invention, the delivery device 12 can be positioned to apply a stimulus (e.g., a vector) to associated white matter 14 into which these and other epileptogenic zones project so as to induce desired physiological changes in target brain cells that project into the white matter where the stimulus is delivered. As described herein, the stimulus can be genetic, pharmacological and/or electrical.

In the case of employing a genetic stimulus, for example, injecting a vector into white matter zone(s) enables retrograde transport of the vector to the target cells of non-white matter regions associated with such zones. This enables expression of therapeutic proteins encoded by genetic materials in the vector. The transfer of genetic materials to the target cells further enables expression of such therapeutic proteins that, in turn, induces a physiological response. The response can modify the phenotypic characteristics of cells and thus change the functional role of neurons and/or alter synaptic activity. As a result, this approach can help improve the patient's quality of life.

In the particular case of epileptoid convulsions originating in the hippocampus, for instance, a vector can be injected into the fornix to alter cell phenotypes in the hippocampus, which can be engineered to express proteins or enzymes capable of mitigating electrical activity associated with seizures. The fornix is the white matter tract that is a major output pathway of each hippocampal formation, connecting it to the frontal lobe, and parts of thalamus and hypothalamus. Thus, mircoinjection of the vector should be precise to affect only the anatomic sources determined to be epileptogenic. Various techniques can be employed to help ensure microinjection into appropriate white matter 14 to affect the desired target cells.

Additionally, a vector or other stimulus can be applied to the corpus callosum, for example, for affecting neocortical areas. The corpus callosum comprises the white matter bundles which collectively serve to interconnect cortical areas in the two cerebral hemispheres of the brain 16. Those skilled in the art will understand and appreciate that other white matter tracts (e.g., the temporal stem) can be utilized to overdrive or modify synaptic activity in other epileptic zones (e.g., lateral or temporal lobes). As mentioned above, one or more delivery devices can be used to supply stimuli to appropriate white matter, such as unilaterally or bilaterally, depending on the epileptogenic zone or zones.

While the foregoing description has mainly focused on a gene therapy applied to white matter to affect synaptic activity in the brain, it will be understood and appreciated that electrical or pharmacological stimulation can be employed in addition to or as an alternative to such gene therapy. For example, the delivery device 12 can also include a stimulator electrically associated with a white matter 14 of a patient's brain 16. That is, the delivery device 12 can include an integrated microneedle and electrical stimulator (e.g., an electrical probe). The delivery system 18 can be operative to supply both a vector solution to the needle portion and an appropriate power source for supplying electrical energy at a desired frequency to the stimulator portion of the delivery device 12. Alternatively or additionally, one or more separate electrical stimulators can be utilized to electrically stimulate the white matter 14 to alter or overdrive electrical activity in non-white matter brain cells that project into the stimulated white matter.

Various configurations of stimulators can be utilized for white matter stimulation in accordance with an aspect of the present invention. For example, the stimulator can be configured as an elongated rod, a depth electrode, a ring, a clamp, or other devices capable of providing desired electrical stimulation to the white matter 14. The stimulator can be self-contained and include a signal generator or, alternatively, it may receive electrical signals from a control system. According to one particular aspect, the stimulator can be collapsible or otherwise deformable to facilitate its endoscopic (or stereotactic) implantation.

In a system that includes an electrical stimulator, a control system 22 (e.g., implemented as part of the delivery system 18) is operative to control operation of the stimulator, such as by providing a signal to the stimulator for electrically stimulating the white matter tract 14 based on the signal. For example, the control system 22 can be coupled to the stimulator through an electrically conductive element, which provides an electrical signal having desired electrical characteristics. Alternatively or additionally, the control system 22 can be configured to activate the stimulator via wireless means, such as electromagnetic fields (e.g., radio frequency (RF)), magnetic fields and the like to provide desired stimulation. That is, a direct connection between the control system 22 and the stimulator is not required.

The control system 22 can include a signal generator (not shown) programmed and/or configured to activate the stimulator for white matter stimulation at a desired intensity (e.g., amperage) and frequency over a predetermined time period. For example, the signal generator can provide electrical pulses at a frequency ranging from about 0.1 Hz to about 5000 Hz. It has been determined some patient's may respond better to low frequency stimulation, such as at a frequency less than about 10 Hz (e.g., in a range from about 0.5 Hz to about 4 Hz). The duty cycle (or pulse width) of such pulses can also be programmable. The amplitude of electrical current may vary based at least in part on the patient's condition and the white matter 14 structure to which the stimulator is positioned. For example, the signal generator 20 can be configured to provide electrical current having an amplitude in a range from 0 to about 5 mA, which can be a monophase or polyphase signal.

By way of further example, the system 10 can be implemented as a closed loop system operative to activate delivery of one or more stimulus to desired white matter 14 in response a sensed characteristic of the brain 16. For example, one or more sensors, indicated at S1 through SN, where N is an integer greater than zero, can be used to sense electrical (or chemical) activity associated with a seizure or other neurological condition. The sensors provide sensor signals to a sensor system 28 indicative of the sensed activity. The sensor system is programmed and/or configured to provide information to the delivery system (or to a physician) indicative of the sensed condition, which information can be employed to adjust parameters associated with stimulus delivery.

The sensors S1-SN can be subdural or external probes located at or near the determined epileptogenic zones. Alternatively or additionally, the delivery device 12 can itself be configured to operate as a sensor and provide signals to the delivery system 18 indicative of seizure activity. The delivery system 18 thus can control the delivery of one or more stimuli (e.g., electrical, pharmacological, gene therapy or a combination thereof) to white matter as a function of sensed electrical (or chemical) activity of the brain 16, such as indicated by the sensor system 28. Those skilled in the art will understand and appreciate various types of sensors S1-SN and detection software (e.g., implemented in the sensor system 28) that can be utilized to detect seizure onset, all of which can be employed to control delivery of one or more stimuli to white matter 14 in accordance with an aspect of the present invention.

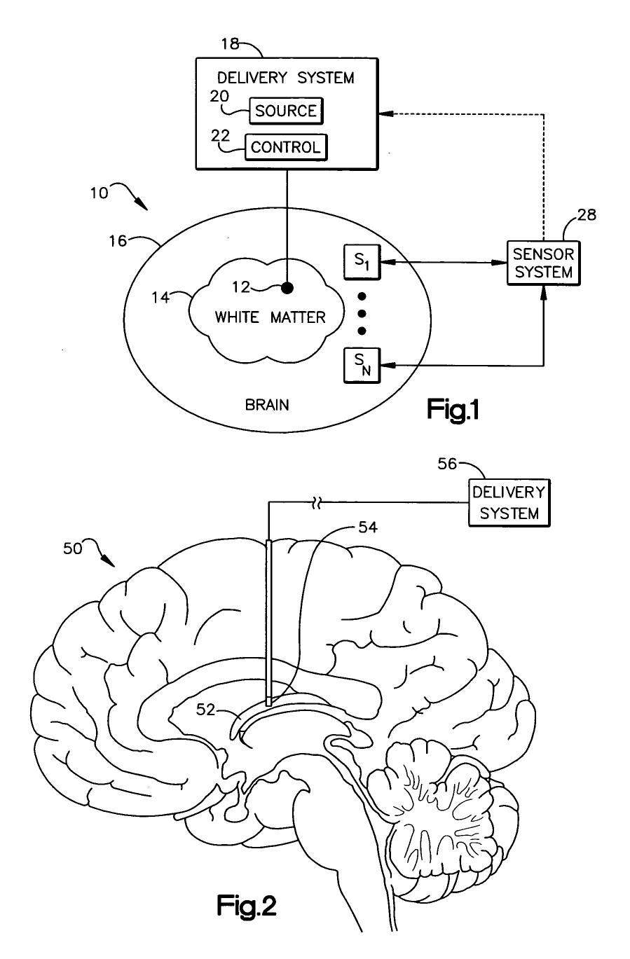

FIG. 2 is an example of a stimulus delivery system 50 implemented to supply one or more stimuli to the fornix 52 in accordance with an aspect of the present invention. In this example, the system 50 includes a stimulus delivery device 54 that is positioned for delivering a stimulus to the body of the fornix 52, such as for corresponding hippocampal stimulation. As mentioned above, the stimulus can be genetic, pharmacological, electrical or a combination of any thereof.

For example, the delivery device 54 can include one or more microneedles operative to deliver a predetermined microinjection of a vector into the fornix 52. An associated delivery system (e.g., a syringe) 56 is operative to supply the vector to the delivery device 54. The vector comprises, for example, genetic material in a solution for inducing expression of a therapeutic protein in target cells (e.g., of the hippocampus) that project into the parts of the fornix 52 where the microneedles are positioned. Such microneedles can also be employed to deliver desired pharmacological agents. Alternatively or additionally, the delivery device 54 can include one or more electrodes that contact the fornix 52 for providing electrical stimulation according to electrical signals from an associated electrical signal generator in the delivery system. Those skilled in the art will appreciate various types and configurations of stimulators that can be utilized to supply a desired electrical stimulus to the fornix, all of which are contemplated as falling within the scope of the present invention.

Those skilled in the art will understand and appreciate that the position of the delivery device relative to the fornix 52 may vary from patient to patient as well as based on the determined location of the epileptogenic focus. As mentioned above, the delivery device 54 can be positioned stereotactically or endoscopically. Endoscopy is particularly useful for positioning the stimulator at the fornix, as the fornix is accessible through corresponding lateral ventricles. Endoscopy thus facilitates implantation of the device 54 aided by its visual component.

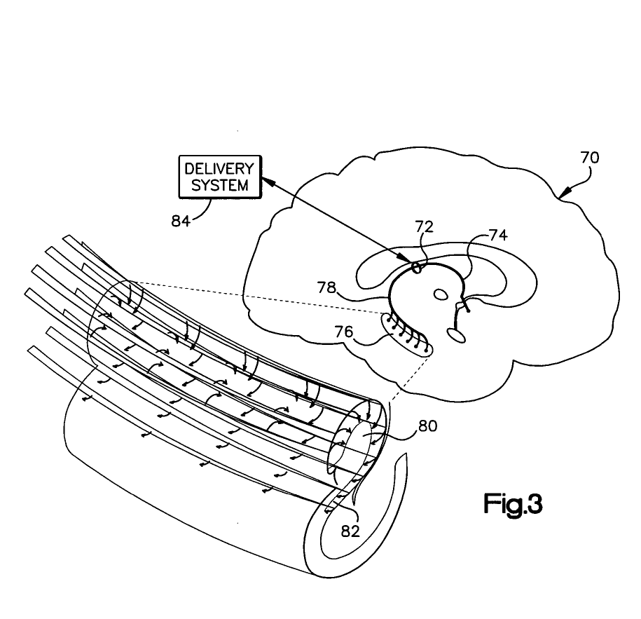

FIG. 3 depicts another example of delivering a stimulus to the fornix in accordance with an aspect of the present invention. In this example, an annular delivery device 72 has been implanted around part of the fornix 74, such as at the fornix body spaced apart from the hippocampus 76. Such implanted delivery device 72 can be configured to provide for delivery of genetic material (e.g., through associated microneedles that engaged selected parts of the fornix), pharmacological agents (also through microneedles), electrical stimulation (e.g., through associated probes or the body of device) or a combination of such agents. A delivery system 84 is operative to provide the suitable stimulus to the delivery device 72.

The fornix 74 includes numerous neuron fibers, schematically represented at 78. Approximately 50% of the fornix fibers 78, which includes fibers for both orthodromic and antidromic impulses, connect the hippocampus 76 with the hyphothalamus (not shown). Such fibers also form part of the circuit of Papez. Because the fornix fibers 78 connect to the hippocampus 76, such fibers provide an efficient pathway for transferring electrical at least a substantial portion of the stimulus from the fornix 74 to the hippocampus 76. The enlarged part of FIG. 3 further diagrammatically represents the transport of the stimulus at the juncture between the crura of fornix 80 to the fimbria of hippocampus 82. It will be appreciated that the resulting physiological effect in the hippocampal will varies based on the type and amount of stimulus supplied to the fornix 74.

Those skilled in the art will understand and appreciate that such fornix stimulation can, in accordance with an aspect of the present invention, provide effective seizure control at focal areas (e.g., the hippocampus 76) directly connected with the associated fibers 78. In the case of gene-based therapy, for example, the fornix fibers 78 provide a suitable pathway for the retrograde transport of a viral or non-viral vector to the target cells in the hippocampus 76. Such fibers also tend to facilitate transfer of electrical signals from the fornix 74 to the hippocampus, in the case of electrical stimulation. The supply of stimulus to the fornix 74 can be controlled by electrical signals provided by an associated signal generator of the delivery system 84, which can be located intra-cranially (e.g., subdurally) or at least a portion of the generator can be exteriorized from the patient.

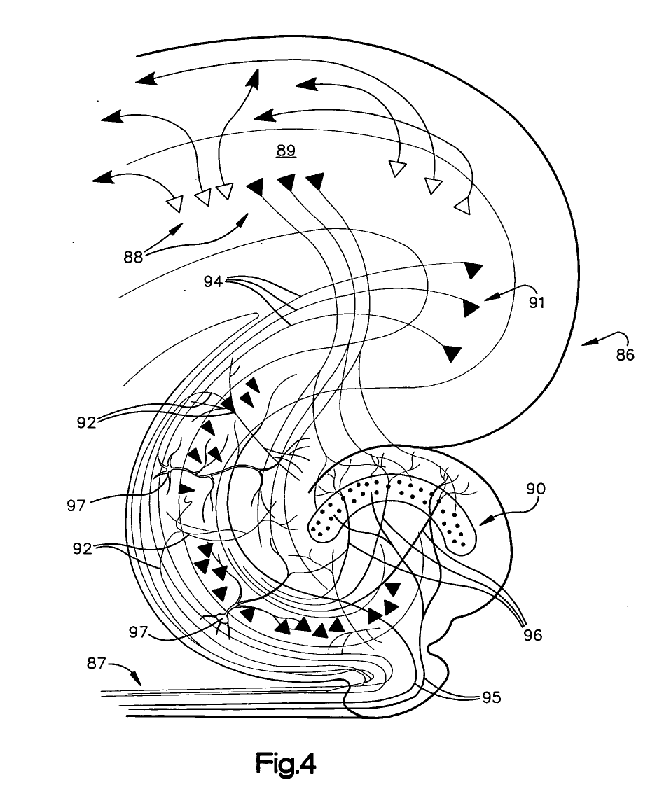

FIG. 4 is schematic representation of the major information pathways in the hippocampal region 86 and their relationship with the fornix 87. From this figure, those skilled in the art will appreciate the types of pathways that can be utilized for transporting a stimulus to epileptogenic zones in the hippocampal region in response to delivering one or more stimuli to the fornix 87 in accordance with an aspect of the present invention.

As represented in FIG. 4, the perforant pathway 88 carries output from the superficial entorhinal cortex 89, which forms the input to the hippocampus and is responsible for the pre-processing of input signals. The perforant pathways 88 carry signals from the entorhinal cortex 89 to the dentate gyrus 90, and information travels thence to fields CA1-CA4, the subiculum 91, and back to the deep layers of the entorhinal cortex, which, in turn, sends output back to the sensory association areas. Also depicted are efferents 92 from pyramidal cells 93 in hippocampal fields CA1-CA4. Efferents 94 from the subiculum 91 are also associated with the fornix 87. Afferents 95 from the fornix 87 are also shown as terminating in the mossy fibers 96 of the dentate gyrus 90. The mossy fibers 96 branch profusely in white matter structures, each branch having multiple swellings that contain round vesicles and synaptic thickenings. Basket cells 97 further are illustrated, which inhibit the piriform neurons, which, in turn, inhibit the deep nuclei and the vestibular nuclei on which their axons synapse.

FIG. 5 illustrates an example of corpus callosal stimulation in accordance with an aspect of the present invention. In this example, the type of stimulus delivery system 150 being utilized is similar to that described above with respect to FIG. 2. Briefly stated, the system 150 includes a delivery device 152 that has a distal end 154 positioned at desired location in selected white matter of the brain 158. An elongate rod 156 (e.g., comprising a microneedle) is inserted into the brain 158 in a minimally invasive manner to position the end 154 of the delivery device 152 in contact with the corpus callosum 160.

The delivery device 152 can include one or more delivery mechanisms capable of delivering predetermined quantities of one or more stimuli at precise locations of the corpus callosum 160. For example, the distal end 154 can correspond to a distal end of a microneedle that receives a vector from an associated delivery system 161. Alternatively or additionally, the delivery device 152 can include one or more electrodes, which are operative to provide electrical stimulation according to electrical signals provided by a signal generator (not shown) of the delivery system 161. As mentioned above, gene therapy supplied to the corpus callosum, pharmacological agents and/or electrical stimulation of the corpus callosum 160 can effectively treat epileptogenic zones in the neocortical areas by altering or overdriving electrical activity at such epileptic zones.

The corpus callosum 160 is white matter that connects significant regions of the two hemispheres of the brain 158. The corpus callosum 160 includes numerous commissual fibers, specific parts of which interconnect the corpus callosum with corresponding regions of cortex. Various parts of the corpus callosum 160 include the rostrum 162, genu 164, body or trunk 166, and splenium 168. For instance, fibers in the splenium 168 interconnect the occipital and posterior temporal cortices on the two sides of the brain 158. Accordingly, one or more stimuli, which can be the same or different types of stimulus, can be supplied to selected parts of the corpus callosum 160 to achieve desired neuromodulation of correspondingly connected neocortical areas in accordance with an aspect of the present invention. As mentioned above, pathways connecting the cortical areas with the corpus callosum enable transport of the supplied stimulus to the target cells. For gene therapy, those skilled in the art will understand and appreciate various mechanisms (e.g., chemical or electrical) that may be employed to facilitate transport of the vector along such pathways. Additionally, numerous diagnostic modalities exist for determining the location of one or more epileptogenic foci, such as the cortical areas connected with the corpus callosum 160.

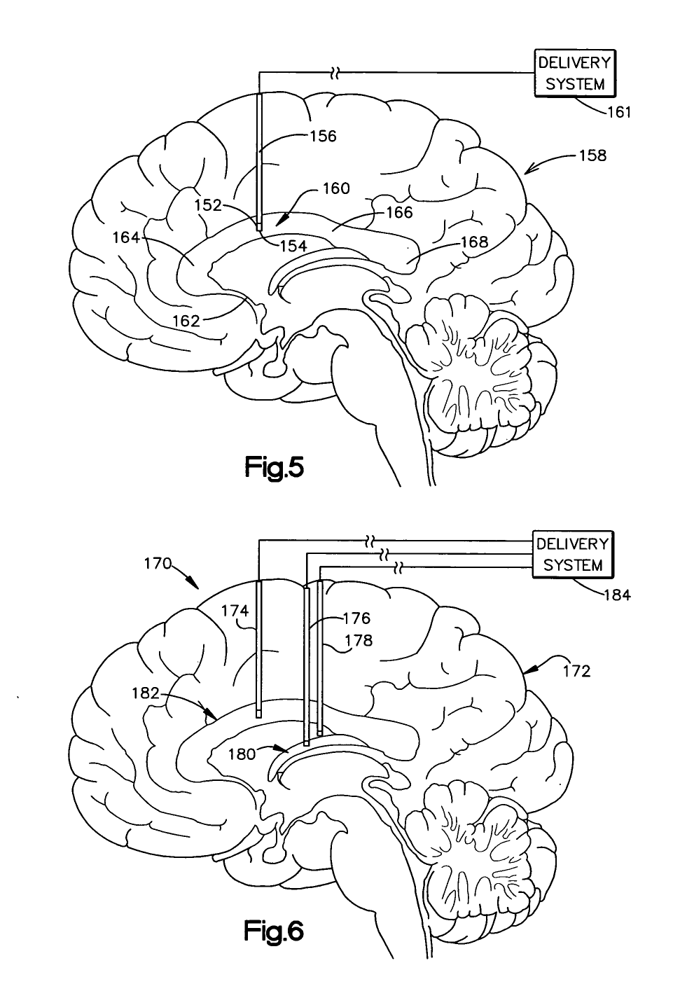

FIG. 6 is an example of another stimulus delivery system 170 operative to supply stimuli to desired parts of plural white matter tracts of the brain 172 in accordance with an aspect of the present invention. In this example, delivery devices 174, 176 and 178 are positioned for selectively supplying stimuli to one or both of the fornix 180 and the corpus callosum 182. While, for purposes of simplicity of illustration, three delivery devices are depicted in FIG. 6, it is to be understood that any number of two or more delivery devices can be utilized in such an approach.

The delivery devices 174, 176 and 178 have been inserted into the brain 172 in a minimally invasive manner (e.g., endoscopically or stereotactically) to position them in contact with desired white matter. In particular, the delivery device 174 is positioned for delivering stimulus to the body of the corpus callosum 182 (e.g., for affecting synaptic activity in associated neocortical regions), whereas the delivery devices 176 and 178 are positioned with their respective distal ends for supplying stimuli in the body of the fornix 180 (e.g., for affecting synaptic activity in hippocampal regions). As mentioned above, the number, types and the precise location of each of the delivery devices 174, 176 and 178 relative to predetermined white matter generally depends on, for example, the location of the epileptogenic foci, seizure frequency and severity or other patient specific parameters.

As mentioned above, a delivery system 184 is operative to selectively supply each of the delivery devices 174, 176, and 178 with one or more corresponding stimuli to induce a desired physiological response in associated tissue that projects into the white matter tracts where the respective devices have been positioned. As described herein, the stimulus can include a vector carrying genetic material for expressing a desired therapeutic protein after transport through pathways connecting the white matter and the target cells. For example, the vector can be engineered for transporting and transferring genes for precursors to neuropeptide transmitters, or for the enzymes that produce or otherwise affect production of neurotransmitters. The particular protein or enzyme generally can be selected according to the symptoms and their severity as well as the desired physiological response.

Additionally or alternatively, the delivery devices 174, 176, and 178 can be configured to supply an electrical stimulus to associated white matter tracts. Such electrical stimulation of white matter stimulates tissue that projects into the white matter by employing the pathways (e.g., neural fibers) to carry electrical signals between the stimulated white matter and the target tissue. A corresponding signal generator thus can be programmed and configured to provide electrical signals (e.g., pulses having desired electrical characteristics, such as described hereinabove) to the stimulator of one or more of the delivery device(s) 174, 176, and 178 for providing desired electrical stimulation.

Those skilled in the art will understand and appreciate the supply of a given type of stimulus at a particular white matter delivery site can be implemented as a predetermined schedule (e.g., open loop configuration) or based on one or more sensed conditions (e.g., closed loop configuration).

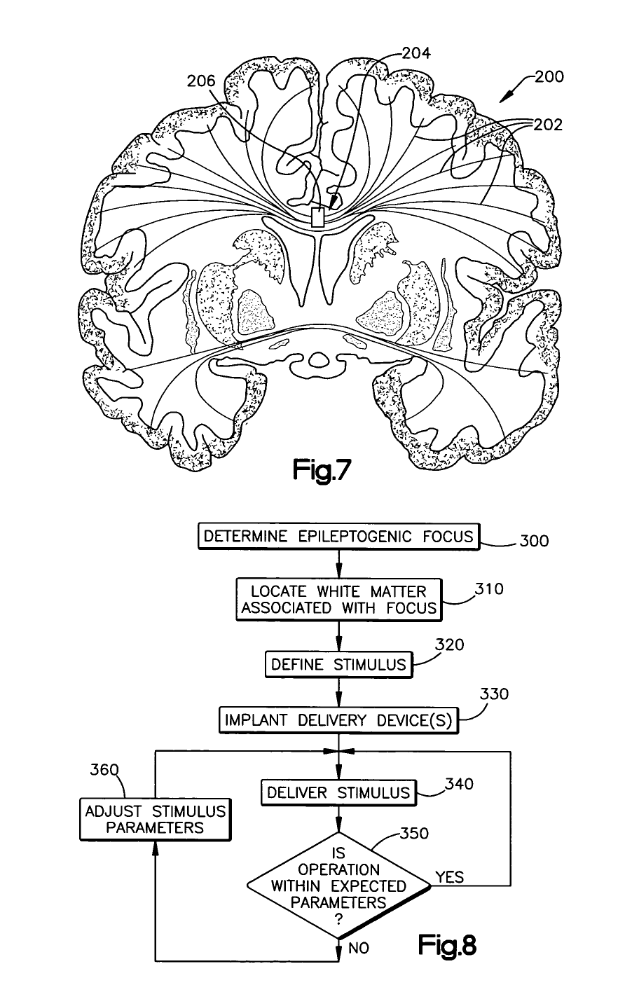

FIG. 7 depicts a coronal section of a brain 200 illustrating white matter fibrous interconnections (or pathways) 202 from the corpus callosum 204 to associated neocortical areas that project into the corpus callosum. A delivery device 206 is positioned in the corpus callosum to facilitate transfer of stimuli from the corpus callosum to the neocortical areas. Thus, by selectively supplying one or more stimuli to different parts of the corpus callosum 174, synaptic activity in corresponding neocortical areas can be modified in desired manners (e.g., overdriven), such as those areas determined to be epileptogenic zones. The treatment can include application one type of stimulus (e.g., gene therapy, pharmacological agent, electrical stimulation) or a combination of different types of stimuli adapted to the patient's evolving circumstances.

In view of the foregoing structural and functional features described above, a methodology for implementing electrical stimulation of white matter tracts, in accordance with an aspect of the present invention, will be better appreciated with reference to FIG. 8. Those skilled in the art will understand and appreciate that not all illustrated features may be required to implement a methodology in accordance with an aspect of the present invention. While, for purposes of simplicity of explanation, the methodology of FIG. 8 is shown and described as being implemented serially, it is to be understood and appreciated that the present invention is not limited to the illustrated order, as some parts of the methodology could, in accordance with the present invention, occur in different orders or concurrently with other parts from that shown and described. Various parts of the methodology can be implemented as computer executable instructions running in a computer or other microprocessor based device (e.g., a signal generator or other associated control system).

The methodology can be performed for patients that have seizures which are intractable to standard treatments such as various anti-epileptic medications. The methodology begins at 300 in which the location of one or more epileptogenic foci is determined. This determination can be made based on one or a combination of diagnostic modalities, such as mentioned herein above. Next, at 310, corresponding white matter associated with or otherwise connected with the epileptogenic focus of foci are located. Such locations can define white matter implantation sites for one or more delivery devices according to an aspect of the present invention.

For example, the fornix can be used if the epileptogenic focus has been determined to be the hippocampus and the corpus callosum can be used as the stimulus delivery site where the epileptogenic focus has been determined to be neocortical. Additionally, perforant pathways can be utilized as a delivery site to selectively transport a desired stimulus to the hippocampus. The implant site can be further selected based on various patient specific parameters, such as mentioned above. One or more delivery devices can be implanted unilaterally or bilaterally depending on the epileptogenic focus or foci.

At 320, the stimulus is defined. The stimulus can comprise supplying a genetic vector, pharmacological agents (e.g., drugs), electrical stimulation or a combination thereof. For gene therapy via white matter, the vector is engineered to include genetic material that encodes a desired therapeutic protein, such as genes for precursors to neuropeptide transmitters, or for the enzymes that produce or otherwise affect production of neurotransmitters. As described herein, gene therapy has an advantage that the genetic material can be engineered to express virtually any protein of interest, many of which may not be capable of effective oral or intravenous adminsitration. Additionally, the vectors can be designed to be cell specific, such as by using tissue-specific promoters in connection with the genes (or gene sequence).

For a stimulus that includes electrical stimulation of white matter, the electrical characteristics generally will vary depending on whether the system is being implemented as an open or closed loop system, a variety or patient specific indications as well as the proximity and electrical pathways interconnecting the white matter and the predetermined epileptogenic zone(s). The pharmacological agents, electrical stimulation and/or other mechanisms that define the stimulus further can be employed to facilitate transport of a vector from the white matter to the associated target cells.

At 330, one or more delivery devices is implanted in white matter for supplying a predetermined stimulus (or plural stimuli) to the regions of white matter determined at 310. As described herein, the white matter delivery site(s) is coupled via neural pathways to associated tissue, such as the source of the disorder or disease being treated (e.g., epileptogenic zone for epilepsy). The delivery device can be implanted stereotactically or endoscopically depending generally on the implant site and type of delivery mechanism being used.

At 340, stimulus delivery is initiated. A stimulus delivery system, which includes a source for supplying a quantity of stimulus, is coupled with each delivery device. Upon activation, the delivery system causes the delivery device to deliver a predetermined stimulus to the appropriate delivery site. The stimulus can be a solution of a genetic vector engineered to induce expression of a desired therapeutic protein. Alternatively or additionally, the stimulus can include an intermittent, periodic or chronic electrical stimulation of the white matter and/or pharmacological agents. A single delivery device can be capable of supplying one or more types of stimuli to the white matter delivery site. Alternatively, separate delivery devices can be employed for delivering different types of stimuli.

For the example of a gene therapy stimulus, the delivery device (e.g., including one or more microneedles) is employed to administer one or more precise microinjections of a vector into the determined white matter delivery site. As mentioned above, the vector can be viral or non-viral and is engineered to transfer genetic material capable of inducing expression of one or more desired therapeutic proteins in the target cells that project into the white matter delivery site. For instance, the genetic material can encode therapeutic proteins of the type capable of increasing inhibitory synaptic activity (e.g., GAD and the like). The vectors themselves further can be designed to facilitate retrograde transport from the white matter through neural pathways to the desired target cells. It is to be appreciated that additional mechanisms (e.g., electrical or chemical), which may form part of the stimulus, also can be employed to facilitate such transport.

For an electrical stimulation example, a signal generator can be configured to provide electrical pulses to one or more electrodes of the stimulator at a frequency ranging from about 0.1 Hz to about 5000 Hz. As mentioned above, low frequency, such as less than about 10 Hz (e.g., in a range from about 0.5 Hz to about 4 Hz) can also be employed. The duty cycle of the electrical pulses also can be programmable. The amplitude of electrical current can be set based at least in part on the patient's condition and the white matter structure being stimulated for overdriving the epileptogenic focus. Electrical current pulses can be provided having an amplitude in a range from about 0 to about 5 mA, which pulses can be monophasic or polyphasic signals, for example. During normal operation, electrical stimulation of the white matter tract results in indirect electrical stimulation of the determined epileptogenic zone via the electrical pathway provided by the white matter structure fibrously connected with the zone, thereby overdriving synaptic activity in the epileptogenic zone that projects into the stimulated white matter.

At 350, a determination is made as to whether operation of the stimulation system is within expected operating parameters. This determination can be made by physician, such as during seizure monitoring using appropriate diagnostic techniques. Alternatively or additionally, the determination can be made by a processor executing a control program, such as part of a closed loop implementation according to an aspect of the present invention. If the determination is positive, indicating that operation is within expected parameters, the methodology can loop back to 340 and continue normal operation. Normal operation, which is dependent on the type of stimulus or stimuli being delivered, can include periodic stimulus delivery or allowing the stimulus to function intracellularly.

If the determination at 350 is negative, the methodology proceeds to 360 in which one or more operating parameters can be adjusted. The adjustments at 360, for example, can be made manually by physician (e.g., reprogramming the stimulation system) to optimize operation for mitigating or helping induce the desired physiological response for the patient. The adjustments can include modifying the administration characteristics or quantity of the stimulus being delivered at 340. Additionally, or alternatively, the adjustment can include providing an additional stimulus via the implanted delivery device, moving the location of one or more implanted delivery devices and/or implanting another delivery device and so forth.

The adjustments at 360 can be based on empirical studies and other data (e.g., patient-specific data or aggregate data collected from a group of patients). Those skilled in the art will understand and appreciate that such adjustments also can be implemented in real time, such as part of the closed loop control process based on feedback from one or more sensors (e.g., intra-cranial or external). The adjustments can also include terminating application of stimulus for an extended period of time or indefinitely, if deemed appropriate.

From 360, the methodology returns to 340 in which normal operation can continue based on the adjustments at 360. For gene therapy implemented in accordance with an aspect of the present invention, it further may be desirable to perform qualitative tests, which can be part of 300 and 310, to help ensure that an adequate pathway exists between the white matter delivery site and the epileptogenic focus. This further can be performed in conjunction with 320 by applying a suitable test stimulus via the implanted delivery device. Once adequate pathways exist for transporting the defined stimulus from the white matter to the target areas, the stimulus can be delivered at 340.

What has been described above includes examples and implementations of the present invention. Because it is not possible to describe every conceivable combination of components, circuitry or methodologies for purposes of describing the present invention, one of ordinary skill in the art will recognize that many further combinations and permutations of the present invention are possible. For example, the above description has primarily focused on treating epileptic seizures, those skilled in the art will understand that it is equally applicable to other types of degenerative diseases and nervous system disorders, such as Parkinson's disease. In view of the foregoing, the present invention is intended to embrace all such alterations, modifications and variations that fall within the spirit and scope of the appended claims.

1. A system for treating a condition in a brain of an animal, comprising:
a delivery mechanism operative to deliver at least one stimulus to a white matter site in the brain that is associated with a non-white matter brain structure, such that delivery of the stimulus to the white matter site induces a physiological response in the associated non-white matter brain structure.

2. The system of claim 1, the stimulus further comprising at least one of a genetic, pharmacological or electrical stimulus.

3. The system of claim 1, the stimulus further comprising a genetic stimulus that includes a vector having genetic material engineered to express a desired therapeutic protein that induces the physiological response.

4. The system of claim 3, wherein the vector is further engineered for retrograde transport from the white matter site to target cells of the associated non-white matter brain structure that project into the white matter site via intervening nerve fibers.

5. The system of claim 3, wherein the vector further comprises at least one of a viral vector and a non-viral vector.

6. The system of claim 3, the physiological response further comprising a modification of phenotypic characteristics of target cells of the associated non-white matter brain structure.

7. The system of claim 1, the non-white matter brain structure further comprising at least one predetermined epileptogenic zone fibrously connected with the white matter site.

8. The system of claim 7, the predetermined epileptogenic zone comprising at least one of a hippocampus, cortical structure, subthalamic nucleus and temporal lobe of the animal.

9. The system of claim 7, the white matter site comprising at least one of a fornix, corpus callosum, perforant path and temporal stem of the animal, the predetermined epileptogenic zone projecting into the white matter site.

10. The system of claim 1, further comprising an electrical stimulator operative to deliver an electrical stimulus to the white matter site to overdrive at least some electrical activity of the associated non-white matter brain structure and thereby induce at least a portion of the physiological response.

11. The system of claim 10, further comprising a genetic stimulus including a vector having genetic materials engineered to express a desired therapeutic protein that induces at least another portion of the physiological response.

12. The system of claim 10, the electrical stimulus further comprising an electrical signal having a frequency that is less than about 10 Hz.

13. The system of claim 12, the frequency being less than or equal to about 3 Hz.

14. The system of claim 1, further comprising a delivery system operative to modify the delivery of the at least one stimulus by the delivery mechanism based on a sensed condition of the animal.

15. A system for changing a physiological condition of an animal, comprising:
a delivery device operative to provide a stimulus to a region of white matter of the animal's brain, the white matter region being connected with a non-white matter region of the animal's brain via a pathway projecting into the region of white matter; and
the stimulus comprising a vector having genetic material engineered to induce expression of at least one therapeutic protein in target cells of the non-white matter region for providing a desired physiological response in the animal.

16. The system of claim 15, wherein the vector is engineered for retrograde transport from the white matter region to the target cells of the non-white matter region that project into the white matter region via the pathway, the pathway comprising intervening nerve fibers.

17. The system of claim 15, wherein the vector further comprises at least one of a viral vector and a non-viral vector.

18. The system of claim 15, the physiological response further comprising a modification of phenotypic characteristics of target cells of the associated non-white matter brain structure.

19. The system of claim 15, further comprising an electrical stimulator operative to deliver an electrical stimulus to the white matter region to at least one of (i) overdrive at least some electrical activity of the associated non-white matter brain structure to induce at least a portion of the physiological response and (ii) facilitate retrograde transport of the vector from the white matter region to the target cells.

20. The system of claim 15, wherein the vector further comprises a pharmacological agent.

21. The system of claim 15, further comprising a control system operative to control at least one of a quantity and frequency of the stimulus being delivered by the delivery device.

22. The system of claim 21, wherein the control system controls the delivery device based on a sensed physiological condition of the animal.

23. A system for treating a condition in a brain of an animal, comprising:
means for locating at least one white matter site of the animal's brain that is fibrously connected with a non-white matter region of the animals brain; and
means for applying a stimulus to the at least one white matter site of the animal's brain to induce a corresponding physiological response in the non-white matter region.

24. The system of claim 23, the stimulus further comprising at least one of a genetic, pharmacological or electrical stimulus.

25. A method comprising:
placing a delivery device in communication with a white matter brain structure that is associated with at least one predetermined non-white matter brain structure; and
using the delivery device to provide a stimulus to the white matter brain structure to induce a desired physiological response in the non-white matter brain structure associated therewith.

26. The method of claim 25, further comprising determining a location of target cells of the non-white matter brain structure and locating the white matter brain structure into which the non-white matter brain structure projects.

27. The method of claim 26, the non-white matter brain structure further comprising an epileptogenic zone.

28. The method of claim 27, the epileptogenic zone comprising at least one of a hippocampus, subthalamic nucleus, cortical structure and temporal lobe.

29. The method of claim 27, further comprising implanting the delivery device at a position for supplying the stimulus into a selected part of at least one of the fornix, corpus callosum and temporal stem to enable transport of at least a portion of the stimulus to the epileptogenic zone.

30. The method of claim 25, the white matter brain structure further comprising at least one of a fornix, a corpus callosum and a temporal stem thereof the brain.

31. The method of claim 25, the stimulus further comprising at least one of a genetic, pharmacological or electrical stimulus operative to induce the desired physiological response.

32. The method of claim 31, the use of the delivery device further comprising applying a genetic stimulus to the white matter structure, the genetic stimulus including a vector of genetic material engineered to express a desired therapeutic protein that induces at least a portion of the desired physiological response.

33. The method of claim 32, the physiological response further comprising a modification of phenotypic characteristics of target cells associated with the non-white matter brain structure based on expression of the therapeutic protein.

34. The method of claim 31, the use of the delivery device further comprising applying an electrical stimulus to the white matter brain structure to at least one of (i) induce at least a portion of the physiological response and (ii) facilitate retrograde transport of the vector from the white matter region to the target cells.

35. The method of claim 25, the use of the delivery device further comprising applying an electrical stimulus to the white matter brain structure to induce at least a portion of the physiological response.

36. The method of claim 35, the electrical stimulus further comprising an electrical signal having a frequency that is less than or equal to about 3 Hz.

37. The method of claim 25, the placement of the delivery device further comprising at least one of stereotaxis and endoscopy to facilitate placing the delivery device in communication with the white matter brain structure.
